(19) United States
(12) Patent Application Publication
(10) Pub. No.: US 2020/0187805 A1
(43) Pub. Date: Jun. 18, 2020

(54) MEDICAL ELECTRODES USING FLEXIBLE CIRCUITS, AND METHODS OF MANUFACTURING

(71) Applicant: NeuroTronik IP Holding (Jersey) Limited, St. Helier (JE)

(72) Inventors: Leslie Purcell, Raleigh, NC (US); Scott Purcell, Raleigh, NC (US)

(21) Appl. No.: 16/712,936

(22) Filed: Dec. 12, 2019

Related U.S. Application Data
(60) Provisional application No. 62/778,353, filed on Dec. 12, 2018.

(51) Int. Cl. A61B 5/042; A61N 1/05; A61B 5/00; B29C 65/60

(52) U.S. Cl. CPC A61B 5/0422; A61N 1/0587; B29L 2031/753; B29C 65/601; A61B 2562/125; A61B 5/6858

(57) ABSTRACT

Methods for joining a flexible circuit to a strut for, for example, use on an electrode array, including connecting the flexible circuit to the array using a rivet and expanding an end of the rivet using heat staking or alternative processes to maintain the connection.

This application claims the benefit of U.S. Provisional Application No. 62/778,353, filed Dec. 12, 2019.

### TECHNICAL FIELD OF THE INVENTION

The present application generally relates electrodes for delivering energy or stimulus to tissue or structure of the body. More specifically, the application relates to electrode manufacturing processes.

### BACKGROUND

Co-pending U.S. application Ser. No. 13/547,031 entitled System and Method for Acute Neuromodulation, filed Jul. 11, 2012 (Attorney Docket: IAC-1260; the “'031 application”), filed by an entity engaged in research with the owner of the present application, is attached at the Appendix and incorporated herein by reference. The '031 application describes a system which may be used for hemodynamic control in the acute hospital care setting, by transvascularly directing therapeutic stimulus to parasympathetic nerves and/or sympathetic cardiac nerves using electrodes positioned in the superior vena cava (SVC). In disclosed embodiments, delivery of the parasympathetic and sympathetic therapy decreases the patient's heart rate (through the delivery of therapy to the parasympathetic nerves) and elevates or maintains the blood pressure (through the delivery of therapy to the cardiac sympathetic nerves) of the patient in treatment of heart failure.

Co-pending U.S. application Ser. No. 14/642,699 (the '699), filed Mar. 9, 2015 and U.S. Ser. No. 14/801,560 (the '560), filed Jul. 16, 2015, each incorporated by reference, describe transvascularly directing therapeutic stimulus to parasympathetic and/or sympathetic cardiac nerves using electrodes positioned in the SVC, right brachiocephalic vein, and/or left brachiocephalic vein and/or other sites. As with the system disclosed in the '031, the methods disclosed in these applications can decrease the patient's heart rate (through the delivery of therapy to the parasympathetic nerves) and elevate or maintain the blood pressure (through the delivery of therapy to the cardiac sympathetic nerves) of the patient in treatment of heart failure.

The '699 and '560 applications describe one form of catheter device that may be used to perform transvascular neuromodulation. In particular, these applications shows a support or electrode carrying member 10 of the type shown in FIG. 1 on the distal part of a catheter member 14. The electrode carrying member 10 includes a plurality of struts 12. One or more of the struts carries one or a plurality of electrodes 17. The electrode carrying member 10 is designed to bias such electrodes into contact with the vessel wall. The material forming the struts 12 may have a shape set or shape memory that aids in biasing the circumferentially-outward facing surfaces (and thus the electrodes) against the vessel wall. The applications describe that the electrodes 17 may be mounted to or formed onto a substrate 15 that is itself mounted onto a strut or a plurality of strut. It is also disclosed that the struts and electrodes may use flex circuit or printed circuit elements.

The present application describes electrode support assemblies in which flexible circuits (having electrodes and/or other components on them) may be mounted to an electrode support.

### BRIEF DESCRIPTION OF THE DRAWINGS

FIG. 1 shows electrode carrying members of the type shown in the '699 and '560 applications, with electrodes carried thereon.

FIG. 2 shows components of an electrode array prior to assembly, and illustrates a method of assembling the flexible circuit to a strut on the array.

FIG. 3A is a side elevation view of a rivet shown in FIG. 2 prior to heat staking.

FIG. 3B shows the rivet of FIG. 3A following heat staking.

FIG. 4A is a side elevation view of a rivet, flexible circuit, and strut assembled prior to heat staking.

FIG. 4B is a side perspective view of the configuration of FIG. 4A.

FIG. 4C is a cross-section view taken along the plane designated B-B in FIG. 4B.

FIG. 5A is a side elevation view of a rivet, flexible circuit, and strut following heat staking.

FIG. 5B is a side perspective view of the configuration of FIG. 5A.

FIG. 5C is a cross-section view taken along the plane designated A-A in FIG. 5B.

FIG. 6A is a bottom plan view showing an embodiment of a rivet having an obround shaft that is suitable for use in the described assembly, and FIG. 6B is a plan view of a strut having an opening shaped to receive the strut of FIG. 6B.

FIG. 6C shows the strut of FIG. 6B with the rivet and flex circuit attached to it.

FIG. 6D is an end view of the strut of FIG. 6C;

FIG. 6E is a side view of the strut of FIG. 6C.

FIG. 7 illustrates an alternative rivet design using a snap-fit configuration.

FIG. 8A is a side elevation view of a flexible circuit and strut assembled using the rivet of FIG. 7.

FIG. 8B is a side perspective view of the configuration of FIG. 8A.

FIG. 8C is a cross-section view taken along the plane designated A-A in FIG. 8B.

FIG. 9 is a perspective view of an electrode array prior to assembly of the flexible circuit onto a corresponding strut and illustrating a flexible circuit with an integrated rivet.

FIG. 10 shows a cross-section of the struts and flexible circuit of FIG. 10, with the layering of the flexible circuit exaggerated for clarity.

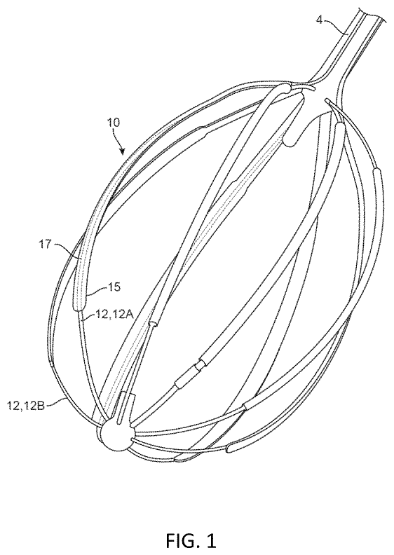

### DESCRIPTION

This application describes methods for joining a flexible circuit to a support. In the given examples, the support is a strut of an electrode array such as a basket type of array, and the flexible circuit is one having electrodes used for delivering therapy and/or sensing, but it should be understood that the types of flexible circuits and supports may be different than the types described here without departing from the scope of the invention. Additionally, the methods and assembly configurations described here are described with reference to an intravascular electrode array electrode, of the type described in the Background, that is used to deliver transvascular therapy to target nerves for acute neuromodulation. However, these concepts may be used for any other types of electrodes used to deliver therapy or sense activity in medical procedures, including without limitation chronically-implantable or acute neuromodulation systems for transvascular nerve stimulation, other types of neuromodulation systems whether or not stimulus is delivered from intravascular sites, or electrophysiology systems for mapping electrical activity of the heart or delivering therapy using stimulation energy or ablation energy using electrodes positioned in the heart, vasculature or elsewhere in the body. Finally, certain materials are named for the components used in the process, but it should be understood that alternative materials known to those skilled in the art for use in such components may be substituted for those that are named.

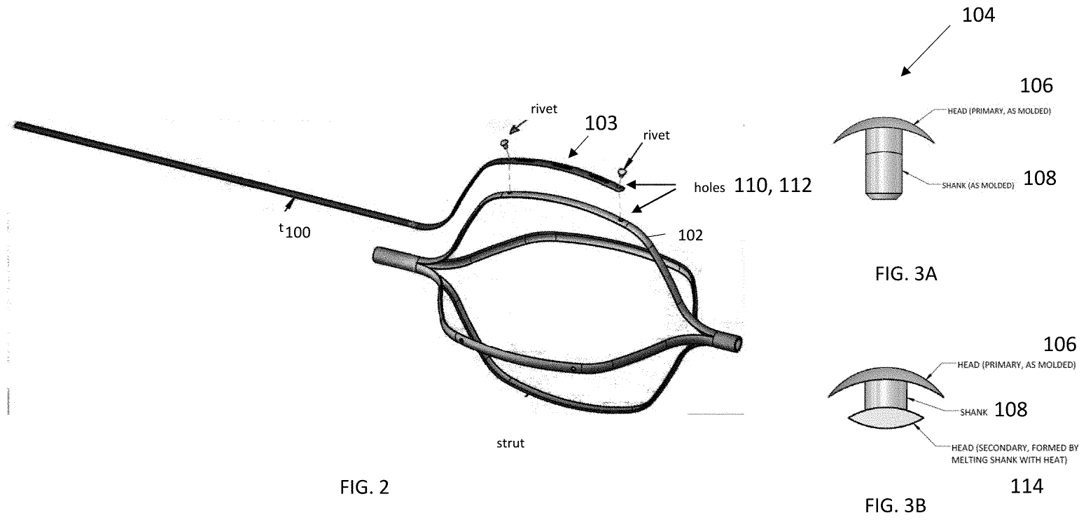

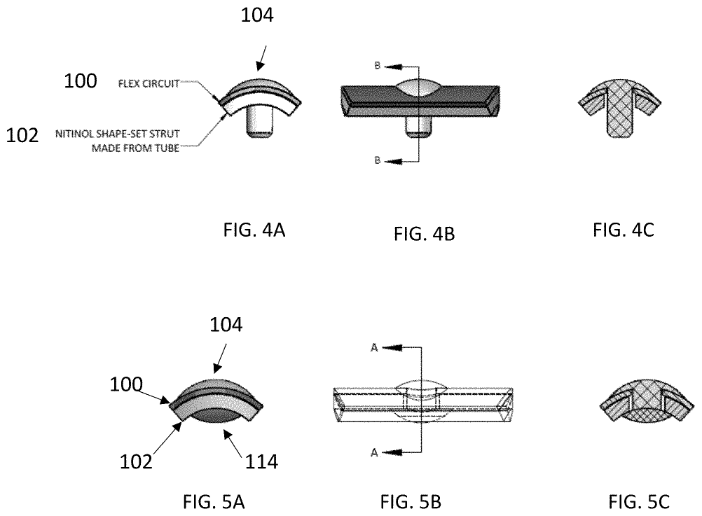

Referring to FIG. 2, the method makes use of a heat staking process to fix a flexible circuit 100 to a strut 102, such as a shape memory strut formed of nitinol or alternative material. The flexible circuit includes the electrodes 103. A side elevation view of a rivet 104 used in this process is shown in FIG. 3A. It includes a primary head 106 on a shank 108. In general, to carry out the method, the shank of the rivet is passed through a hole 110 in the flexible circuit and an aligned hole 112 in the strut as indicated in FIG. 2. FIGS. 4A-4C illustrate this assembly prior to heating.

The rivet is then subjected to a heating process that causes the end of the shank to expand radially and compress longitudinally, forming a secondary head 114 on the opposite end of the shank from the first head. The modified shape of the rivet is shown in FIG. 3B, and the assembly following heat staking is shown in FIGS. 5A-5C. The flexible circuit and strut are thus fixed to one another, captured between the primary head and secondary head of the rivet.

It will be observed with respect to FIGS. 4A-5C that the strut may be one having a cross-section that is arcuate in the lateral direction. The strut may be formed using a process in which an elongate tube is longitudinally cut to form the strut. When this strut shape is used, the primary head of the rivet may be designed with a concave lower face that contacts the convex surface of the strut, as shown in FIG. 3A. As also shown in FIG. 3A, in this embodiment the cross-sectional wall thickness of the primary head may be tapered, with the thinnest region being along the perimeter of the primary head. Following heat staking, the resulting secondary head is nested within the concave curvature of the adjacent face of the strut, creating a smooth transition between the secondary head and the strut. It may also include a convex outer face. See FIGS. 5A and 5C.

In alternative embodiments of the method, the rivet may initially include only a shank, in which case the heat staking process is used to form heads on opposite ends of the shank. As yet another alternative, the rivet may have a primary head during staking, but that primary head may be further deformed during the staking process.

The rivet is preferable formed of a polymeric material, such as nylon (e.g. Nylon 12). A preferred rivet is molded into the shape illustrated in FIG. 3A.

The rivet may also include features that enhance its visibility on a fluoroscopic image, allowing it to be used as a radiopaque marker. This is accomplished by doping the resin used to form the rivet with a radiopaque filler such as Tungsten prior to molding. In some embodiments, a combination of filled and un-filled rivets may be used to form a pattern visible and recognizable under fluoroscopy, to aid the practitioner in understanding the position and orientation of the electrode assembly within the patient. This is further discussed in co-pending U.S. application Ser. No. ______, filed Dec. 12, 2019 (Attorney Ref: NTK2-1510) entitled Fluoroscopic Markers for Single View Positioning, which is incorporated herein by reference.

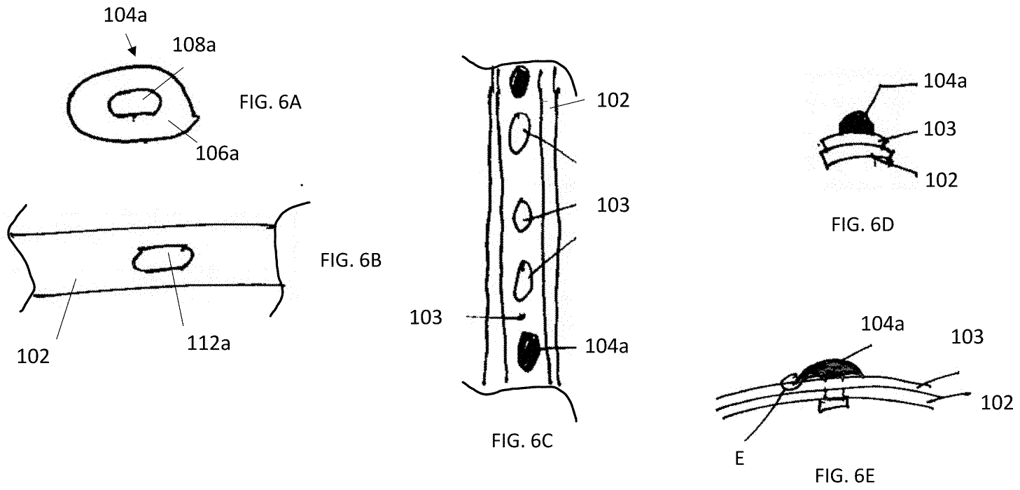

FIG. 6A shows an alternative embodiment of a rivet 104a, which includes a shaft 108a having an obround shape and a head 106a also having an obround shape. The corresponding mounting holes 112a in the strut 102 are likewise obround as shown in FIG. 6B. The long dimension mounting holes is oriented longitudinally relative to the long axis of the strut, and the short dimension oriented laterally. When assembled, the long dimension of the rivet is likewise oriented longitudinally relative to the long axis of the strut, as shown in FIG. 6C. The non-circular shape of the shaft and opening prevents rotation of the rivet relative to the strut.

During use of the catheter, the struts are moved to a radially compressed position and a thin-walled tube is advanced over the struts to maintain them in the compressed position for delivery into a patient's vasculature. As best understood by comparing FIG. 6D (which provides an end view of the assembled strut, flex circuit and rivet) with FIG. 6E (showing the side view), this orientation improves resistance to such thin tubes getting under the leading edge E of the rivet.

In one embodiment, the process is used for an intravascular catheter electrode array formed using multiple struts (e.g. four struts), two of which have flex circuits, and two rivets are used with each flex circuit.

Assembly using the methods described in this application may be performed using a resistance welder (e.g. Amada Miyachi Resistance Welder). It will be understood that given the extremely small size of the rivets and holes, the process is preferably performed under magnification. Vacuum-assisted fixturing and a thermode tailored to the shape and dimensions of the rivet and associated components are also useful for carrying out the process.

In accordance with the process using this equipment, the following steps may be performed in the assembly process:
1. Place 1 rivet, primary head down, on the fixture at the custom-shaped vacuum port (to ensure rivet retention and orientation).
2. Lay the flexible circuit over the rivet, with the rivet shank passing through the hole in the flexible circuit.
3. Lay the strut over the flexible circuit, with the rivet shank passing through the strut hole.
4. Engage the fixture's retention clips over assembly to restrain the assembly.
5. Activate the resistance welder using a pre-programmed heating/cooling cycle. In this step, the thermode lowers onto the rivet shank and melts end of shank to form the secondary head. The thermode is one configured to automatically lower and raise based on time and temperature.
6. Remove the retention clips.
7. Remove the strut from the fixture.
8. Repeats steps 1-7 for the remaining rivets of the catheter array noting hole location to determine which rivet material (filled or unfilled) to use.

### Alternative Embodiments

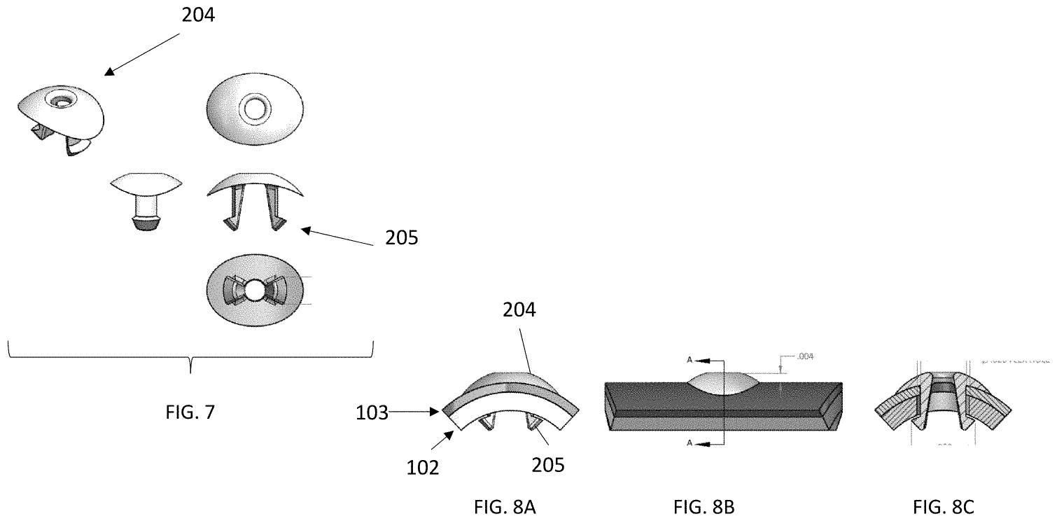

FIG. 7 shows an alternative rivet configuration 204 designed to be assembled using a snap-fit joint method. During use of this design, the rivet would sandwich the flexible circuit to the strut in a manner similar to the heat-staked rivet design discussed above. In this case, however, the rivet would pass through coaxial holes in the strut and the flex circuit until the snap's tangs 205 are engaged. See FIGS. 8A-8C. This cantilevered snap-fit design would be permanent/inseparable once the tangs are engaged. This design would eliminate equipment such as the resistance welder, and eliminate the need for elevated temperatures.

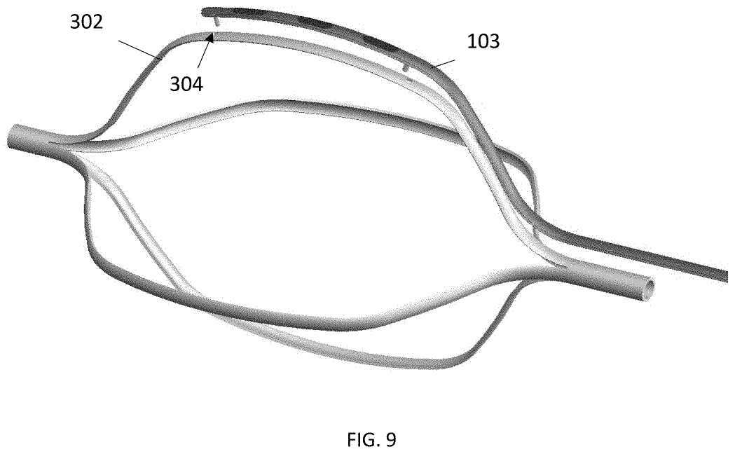

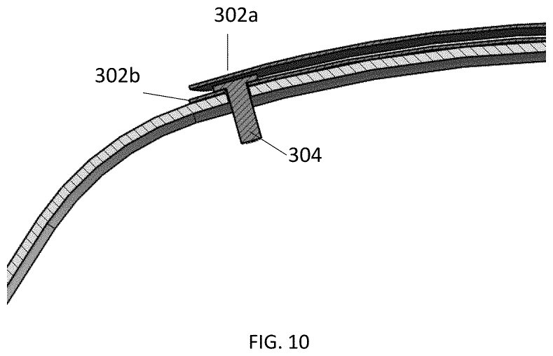

In another alternative embodiment, a flexible circuit 302 is provided that has rivets 304 and/or radiopaque markers embedded into it during initial flexible circuit construction. For example, the rivets and/or markers could be placed between the layers 302a, 302b in the flexible circuit as shown in FIGS. 9 and 10, making the added components part of an inseparable assembly. This would facilitate catheter manufacturing, reduce overall component count and ensure a smooth exterior surface on the flex circuit.

The disclosed methods for applying flex circuits to supports and the resulting assemblies provide a number of advantages over prior art methods and assemblies. In particular, they allow formation of the assembly without the use of glue joints or heat shrink tubing, they improve manufacturability for higher volume production, they avoid creating of particles. Also, the riveted assemblies conform to the unique curved shape of shape memory nitinol strut, and they result in the formation of features on the assembly that can be used as radiopaque markers that can the practitioner can use as a reference using the electrodes while positioning and orienting the electrodes at the relevant anatomy while viewing the procedure on a fluoroscopic image.

All patents and patent applications referred to herein, including for purposes of priority, are incorporated herein by references for all purposes.

1. A method of assembling a flexible circuit and a support positionable in a patient for delivering therapy or sensing electrical activity, the method comprising the steps of:
positioning a flexible circuit in contact with a support;
passing a shaft through the flexible circuit and support, the shaft having a first end and a second end; and
causing the flexible circuit and support to be retained between the first and second ends.

2. The method of claim 1, wherein the method includes passing the shaft through corresponding holes in the support and flexible circuit, and wherein the causing step causes at least one of the first and second ends to be expanded to a diameter larger than the diameter of the holes.

3. The method of claim 2, wherein the causing step includes applying heat to said at least one of the first and second ends.

4. The method of claim 2, wherein the first end includes a primary head, wherein the passing step includes positioning the primary head in contact with the flexible circuit or the support, and wherein the causing step includes expanding the second end to form a secondary head.

5. The method of claim 1, wherein the first end includes a primary head, wherein the second end includes at least two spring members resiliently biased in a radially-outward direction, wherein the passing step includes positioning the spring members in a radially-inward compressed for insertion through the holes, positioning the primary head in contact with the flexible circuit or the support, and releasing the spring members from the compressed position, allowing the spring members to expand in a radially outward direction.

6. The method of claim 1, wherein the shaft is formed of a radiopaque material.

7. A method of assembling a flexible circuit and a support positionable in a patient for delivering therapy or sensing electrical activity, the method comprising the steps of:
positioning a flexible circuit in contact with a support, the flexible circuit including a shaft extending therefrom;
passing the shaft through the support, the shaft having a free end; and
expanding the free end to retain the flexible circuit to the support.

8. The method of claim 7, wherein the method includes passing the shaft through a hole in the support, and wherein the causing step causes the free end to be expanded to a diameter larger than the diameter of the hole.

9. The method of claim 8, wherein the causing step includes applying heat to the free end.

10. The method of claim 8, wherein the free end includes at least two spring members resiliently biased in a radially-outward direction, wherein the passing step includes positioning the spring members in a radially-inward compressed for insertion through the hole, and releasing the spring members from the compressed position, allowing the spring members to expand in a radially outward direction.

11. The method of claim 8, wherein the shaft is formed of a radiopaque material.

12. A support assembled using any of the methods of claim 1, wherein the support is an elongate strut having an arcuate lateral cross section.

13. The support of claim 12 wherein the shaft is a rivet having a primary head including a rivet with a concave surface positionable in contact with the convex face of the strut.

14. The support of claim 12 wherein causing expansion of an end of the shaft results in formation of a head having a convex surface in contact with the concave face of the strut.
